(19) United States
(12) Patent Application Publication
(10) Pub. No.: US 2005/0208531 A1
(43) Pub. Date: Sep. 22, 2005

(54) Biochip substrate and process for its production

(75) Inventors: Hideo Nomoto, Yokohama-shi (JP); Kenji Ishizeki, Yokohama-shi (JP); Shin Tatematsu, Yokohama-shi (JP); Masayuki Ishimaru, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: ASAHI GLASS COMPANY LIMITED, Tokyo (JP)

(21) Appl. No.: 11/003,417

(22) Filed: Dec. 6, 2004

(30) Foreign Application Priority Data
Dec. 9, 2003 (JP) 2003-410803

(51) Int. Cl. H01L 21/00; C12Q 1/68; C12M 1/34

(52) U.S. Cl. 435/6; 435/287.2; 438/1

(57) ABSTRACT

The object of the present invention is to provide a biochip substrate which affords accurate and sensitive measurements using a biochip by enhancing the S/N ratio of the fluorescence from each spot. A biochip substrate for producing a biochip by immobilizing a biological high-molecular-weight oligomer, which substantially reflects fluorescence on the surface on which the oligomer is to be immobilized. Preferably, a biochip substrate which consists essentially of a support and a coating formed on the surface of the support on which the biological high-molecular-weight oligomer is to be immobilized, wherein the coating comprises a reflective film which has a higher refractive index than the support.

### BACKGROUND OF THE INVENTION

1. Field of the Invention

The present invention relates to a substrate suitable to make a biochip by arranging and immobilizing trace amounts of biological high-molecular-weight oligomers such as DNA, RNA, sugar chains and proteins corresponding to hundreds to tens thousands of genes.

2. Discussion of Background

Among biochips, typical are DNA chips having from hundreds to thousands of microspots of numerous DNA fragments immobilized on the substrate. DNA chips are reacted (hybridized) with DNA from human or animals to be examined for numerous DNA sequences to analyze sequence variation among individuals, gene expression in cells in different states and the like. The subsequent description will deal with DNA as a representative, though it also applies to RNA, proteins and sugar chains.

Biochip fabrication is roughly classified on the basis of the method of immobilization of DNA on the substrate, as the photolithographic synthesis on a solid phase and as the array stamping of numerous kinds of pre-synthesized DNA to be tested for on the substrate. In either method, immobilized DNAs (hereinafter referred to as probes) are reacted with a mixture of DNA fragments (hereinafter referred to as analytes). The analytes are usually detected by preliminarily tagging them with fluorophor molecules and measuring the relative fluorescence intensity of each spot under excitation light with a fluorescent reader.

The fluorescence is weaker than the excitation light, and the maximum and minimum analyte densities in a DNA sample differ by 1000 to 10000 times. Low density analytes are difficult to detect accurately because of the noise such as the fluorescence emission or reflection from the support surface and dirt on it.

As an approach for higher ratios of fluorescence intensity to noise (S/N ratios), it was proposed to prevent mishybridization by designing orthogonality with little sequence similarity among probes (Patent document 1). However, this approach has problems such as costly probe design and restricted probe choices. A support having a film with high probe affinity which enables accurate spot formation was proposed to increase fluorescence densities (Patent document 2). However, there is a problem that films with high probe affinity cost a lot to develop and produce with quality control which ensures film homogeneity over the support surface. Further, a frosted finish on the support surface was proposed to increase the surface areas of spots (Patent document 3). However, still higher S/N ratios are demanded.

Provision of a reflective film on the back surface of a support with the opposite surface to be used for analyte immobilization was also proposed to make biochip supports that increase fluorescence readings by theoretically doubling the excitation light which strikes the fluorophor molecules and the fluorescence emitted towards the photo detector. However, this approach is limited to transparent supports and does not improve the S/N ratio as much as theoretically expected because part of the light enters the substrate. Further, formation of an exteriorly reflective vertical wall surrounding each spot on a flat support was proposed to enhance the fluorescence from spots by cutting off noise from the outside (Patent document 4). However, because the reflected fluorescence is not directed straight to the fluorescence sensor of the reader, this approach cannot enhance the fluorescence intensity sufficiently.
[Patent document 1] JP-A-2003-99438 (pages 1-3)
[Patent document 2] JP-A-2003-14744 (pages 1-5)
[Patent document 3] JP-A-2003-107086 (pages 1-4)
[Patent document 4] JP-A-2002-122596 (pages 1-6)

### SUMMARY OF THE INVENTION

The object of the present invention is to provide a biochip substrate which affords accurate and sensitive measurements using a biochip by enhancing the S/N ratio of the fluorescence from each spot.

The present invention provides a biochip substrate for producing a biochip by immobilizing a biological high-molecular-weight oligomer, which substantially reflects fluorescence on the surface on which the oligomer is to be immobilized. According to another aspect of the present invention, the present invention also provides a biochip substrate for producing a biochip by immobilizing a biological high-molecular-weight oligomer, which consists essentially of a support and a coating formed on the surface of the support on which the biological high-molecular-weight oligomer is to be immobilized, wherein the coating comprises a reflective film which has a higher refractive index than the support. According to a still another aspect of the present invention, the present invention provides a biochip substrate which consists essentially of a metal support and an organic film formed on the surface of the metal support on which biological high-molecular-weight oligomer is immobilized. According to a still further aspect of the present invention, the present invention provides a process for producing a biochip substrate which comprises forming a reflective film on the surface of a support on which a biological high-molecular-weight oligomer is to be immobilized, wherein the reflective film has a higher refractive index than the support.

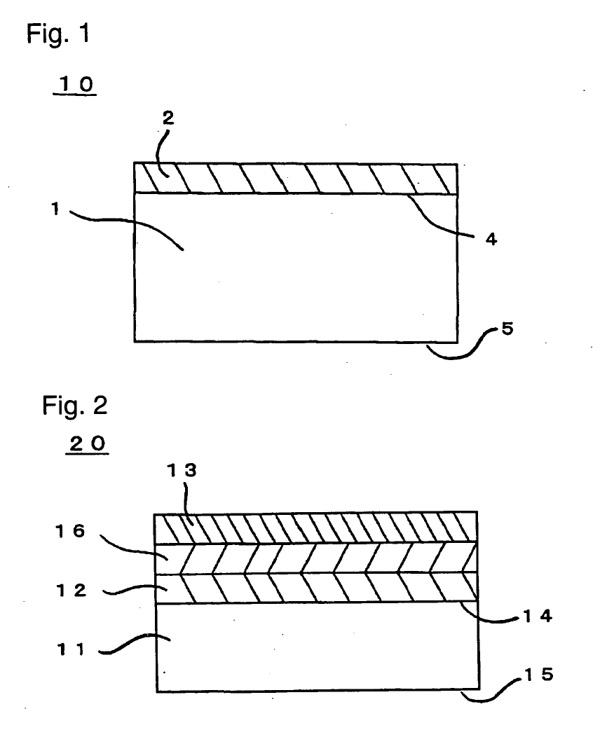

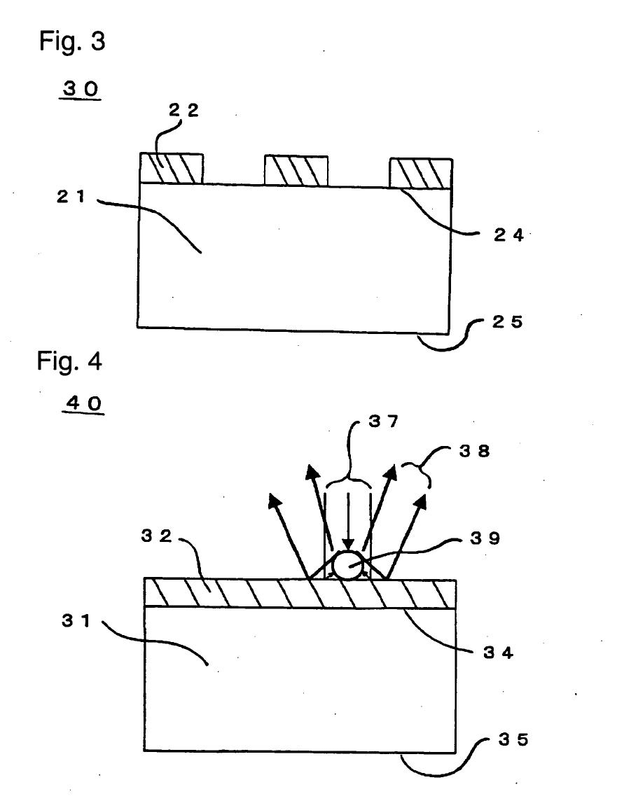

### BRIEF DESCRIPTION OF THE DRAWINGS

[FIG. 1] A cross-sectional view showing the structure of a substrate of the present invention.

[FIG. 2] A cross-sectional view showing the structure of a substrate having an organic film of the present invention.

[FIG. 3] A cross-sectional view showing a structure partly having a reflective film.

[FIG. 4] An illustrative scheme showing how to use a biochip having a substrate of the present invention.

### EXPLANATION OF THE REFERENCE NUMERALS

1, 11, 21 and 31: supports
2, 12, 22 and 32: reflective films
4, 14, 24 and 34: the immobilization surfaces of supports
5, 15, 25 and 35: the back surfaces of supports
10, 20, 30 and 40: substrates of the present invention
13: an organic film.
16: a SiO₂ film
37: excitation light
38: fluorescence
39: an analyte linked to a fluorophor molecule

### DETAILED DESCRIPTION OF THE PREFERRED EMBODIMENTS

The biochip substrate of the present invention enables the fluorescence from the fluorescently tagged analytes to be detected with a high fluorescence intensity by reflecting the fluorescence on the surface of the substrate where the assay targets are immobilized, not on the back surface, and gives high S/N ratios by reducing the noise attributable to the fluorescence from where the immobilized analytes are not present (hereinafter referred to as a background) or to dirt on the immobilization surface. The support does not have to be transparent, and an opaque support may be used.

When a reflective film is present in the present invention, use of a reflective film containing at least one member selected from the group consisting of TiO₂, Ta₂O₅, ZrO₂, Al₂O₃, Si₃N₄, TiNₓ (0<X≦1), Cr, Ag and Ge makes the above-mentioned effects remarkable. Especially, when a film containing SiO₂ and an organic film containing an organic substance having high affinity for the assay targets are formed on the reflective film in this order, the S/N ratio becomes particularly high.

Because such a high S/N ratio is attained, accurate data can be obtained without special changes in the conventional biochip assay procedures and equipment. Further, it is possible to precisely measure fluorescence with a very low intensity from a low density analyte called low expression on which precise information has hardly been obtained conventionally. Because a high S/N ratio can be attained easily, it is possible to obtain more integrated biochips with higher densities by reducing the diameter of each sample spot.

Herein, a biochip substrate means a substrate which is used after a biological high-molecular-weight oligomer is immobilized directly on it or on an organic film formed on it. The biochip substrate of the present invention (hereinafter referred to as the present substrate) is a biochip substrate for producing a biochip by immobilizing a biological high-molecular-weight oligomer which substantially reflects fluorescence on the surface on which the oligomer is to be immobilized. By substantially reflecting fluorescence on the surface on which the oligomer is to be immobilized, it is meant that the fluorescence reflected from the immobilization surface is stronger than the fluorescence reflected from the opposite surface (hereinafter referred to as the back surface) by at least several times.

The support which serves as the base of the present substrate is not particularly limited as long as it is flat. As the material, various types of glass, synthetic quartz glass, ceramics, metals, plastics and the like may be used singly or in combination. In particular, various glasses, synthetic quartz glass and metals are preferred because the fluorescence emission from the support is so little under the excitation light around from 500 nm to 650 nm which is usually used for biochips that the support has little influence on the S/N ratio.

Glass supports are preferable for their flatness. The glass may be produced by any processes without particular restrictions, and the float process is preferred. Glass sheets obtained by the float process are typical examples of the support. Plastic supports are also preferable for their easy and economical availability. The immobilization surface of the support may have any surface profile without particular restrictions, and a support as flat as possible over areas of about 10 to 500 μm in diameter corresponding to single points of measurement on a biochip is preferred.

It is preferred that the substrate of the present invention consists essentially of a support and a coating formed on the immobilization surface of the support, and the coating comprises a reflective film having a higher refractive index than the support, because the immobilization surface reflects fluorescence well. When a reflective film is present, a nonmetal support is preferred because the effect of the reflective film is remarkable. Preferable examples are glass and plastics.

When the reflective film has a higher refractive index than the support, a biochip substrate allowing high S/N ratios can be obtained because most of the fluorescence emitted from assay targets towards the support is reflected by the reflective film.

The cross-sectional view in FIG. 1 shows the structure of a substrate having a reflective film of the present invention. In the figure, 1 denotes a support, 2 denotes a reflective film, 4 denotes the immobilization surface of the support, 5 denotes the back surface of the support, and 10 denotes a substrate of the present invention. FIG. 4 illustrates how to use the biochip of the present invention. In the figure, 31 denotes a support, 32 denotes a reflective film, 34 denotes the immobilization surface of the support 31, 35 denotes the back surface of the support 31, 37 denotes excitation light, 38 denotes fluorescence, 39 denotes a fluorescently tagged analyte, 40 denotes a substrate of the present invention. It illustrates reflection by the reflective film 32 of the fluorescence emitted from the analyte molecule toward the support.

The reflective film 2 in the substrate of the present invention is preferably an inorganic film containing a metallic element because the fluorescence and the excitation light are easily reflected, though it is not particularly restricted as long as it has a higher refractive index than the support. The metallic element is preferably Ti, Ta, Zr, Al, Si, Cr, Ag or Ge. The reflective film containing a metallic element preferably contains at least one member selected from the group consisting of at least one member selected from the group consisting of TiO₂, Ta₂O₅, ZrO₂, Al₂O₃, Si₃N₄, TiNₓ (0<X≦1) Cr and Ag to facilitate formation of a reflective film having a high refractive index. More preferably, Ge is included in the above-mentioned group. In other words, it is more preferable that the reflective film contains at least one member selected from the group consisting of TiO₂, Ta₂O₅, ZrO₂, Al₂O₃, Si₃N₄, TiNₓ (0<X≦1), Cr, Ag and Ge. Especially, a TiNₓ film or a Ge film is the most suitable because fluorescence is weak under excitation light around from 500 nm to 650 nm which is usually used for biochips.

As to the properties of the reflective film 2, it preferably has a refractive index of at least 1.6, more preferably at least 1.8. It is further preferred to differ from the support 1 in refractive index by at least 0.2.

The thickness of the reflective film 2 is preferably from 5 to 200 nm, more preferably from 10 to 100 nm, particularly preferably from 20 to 60 nm, when measured with a metal film thickness meter. The reflective film 2 is thinner than 5 nm, the desired optical properties such as refractive index and reflectance are hardly obtained. If it is thicker than 200 nm, the film may be so uneven that measurement errors may arise, and it is not beneficial in view of cost. The reflective film 2 is not restricted to a monolayer film and may be a multilayer film. In the case of a multilayer film, the thickness is defined as the total thickness. The reflective film 2 may cover the entire immobilization surface or part of the immobilization surface. FIG. 3 is a sectional view showing an embodiment which is partly covered by the reflective film 2. In the figure, 30 denotes the support of the present invention, 21 denotes a support, 22 denotes a reflective film, 24 denotes the immobilization surface of the support 21, and 25 denotes the back surface of the support 21.

The reflective film 2 preferably has a surface roughness Ra of less than 500 nm because analytes are treated evenly on a biochip, and the fluorescent signals from analytes can be read without changing the focus. The surface roughness Ra of the reflective film 2 is more preferably less than 100 nm, in particular less than 50 nm.

The reflective film 2 may be formed by any methods for example, by dry methods such as sputtering, vapor deposition and chemical vapor deposition (CVD) or by wet methods such as spreading and dipping. Sputtering and vapor deposition are preferred to coat only one surface with a material. Sputtering is particularly preferred to achieve an even thickness. It is particularly preferred to carry out the coating in a clean room of class 10000 or less in order to prevent contaminants which can cause measurement errors from adhering.

The present substrate 10 is preferred to further has an organic film 13 having affinity for biological high-molecular-weight oligomers as analytes on the reflective film 2 because analytes are immobilized on the present substrate firmly. As the organic film 13, poly-L-lysine films, aminosilane films and films containing functional groups such as aldehyde groups and carboimide groups on the surface are preferred for their high affinity for biological high-molecular-weight oligomers.

The organic film 13 is preferably from 1 to 50 nm thick. The organic film 13 is not restricted to a monolayer film and may be a multilayer film. In the case of a multilayer film, the thickness is defined as the total thickness. The organic film 13 is preferably formed by dipping or spreading.

Further, when the organic film 13 is used, it is preferred to form a film 16 composed mainly of SiO₂ (hereinafter referred to simply as a SiO₂ film) between the reflective film 12 and the organic film 13 because the organic material is readily bound or electrostatically adsorbed to the polar groups present on the surface such as hydroxyl groups. It is particularly preferred that the SiO₂ film is composed substantially of SiO₂ because the above-mentioned effect becomes more remarkable. The SiO₂ film 16 is preferably from 1 to 50 nm thick. The SiO₂ film 16 is not restricted to a monolayer film and may be a multilayer film. In the case of a multilayer film, the thickness is defined as the total thickness. The SiO₂ film 16 may be formed by sputtering, vapor deposition, CVD, spreading or dipping, preferably by sputtering in view of thickness evenness. FIG. 2 is a cross-sectional view showing the structure of the present substrate 20 obtained by forming a reflective film 12, a SiO₂ film 16 and an organic film 13 successively on a support 11.

In the present substrate 10, another film may be formed between the support 1 and the reflective film 2 to improve adhesiveness. Further, films may be formed on the immobilization surface 4 and the back surface 5 of the support 1 for other purposes than imparting reflectivity such as preventing deformation and imparting discrimination.

An embodiment of the present substrate 10 has a structure obtained by forming a reflective film containing TiNₓ (0<X≦1) or Ge on a support 1 made of a glass material or a plastic, forming a SiO₂ film thereon, and forming an organic film containing aminosilane on the SiO₂ film.

The present substrate may has another structure consisting essentially of a metal support and an organic film formed on the surface of the metal support on which the biological high-molecular-weight oligomer is to be immobilized. The immobilization surface of the metal support is preferred to be flat, especially specular. As the organic film, those mentioned previously to be formed on the reflective film are preferably employed.

### EXAMPLES

Now, examples of the present invention will be given below.

### Example 1

To a mixture of 2.55 g of Ti(OC₄H₉)₄, 17 g of ethanol and 0.75 g of acetylacetone, 1.1 g of 0.1 mol/dm³ aqueous nitric acid was added dropwise with sufficient stirring. Then, the resulting mixture was stirred at room temperature for 1 hour to make a coating solution. A well-washed glass slide (manufactured by Matsunami Glass) having a thickness of 1 mm, a flatness of 50 μm and a refractive index of 1.5 was dipped in the coating solution for 20 seconds and withdrawn at a rate of 24 cm/min to form a film. It was dried at 120° C. for 15 minutes, baked at 550° C. for 30 minutes to obtain a substrate having a TiO₂ film on the surface (hereinafter referred to as the present substrate A). The TiO₂ film had a thickness of 100 nm, a surface roughness Ra of 50 nm, a refractive index of 2.4 and a surface resistance of 2×10¹⁰ Ω/□. For comparison, a glass slide coated with poly-L-lysine (hereinafter referred to as comparative substrates) was prepared.

For comparison of the fluorescence characteristics of the present substrate A and the comparative substrate, DNA fragments (75-mer) linked to fluorophor labels (Cy3 and Cy5) were diluted with distilled water to make DNA dilutions ranging from 0.1 to 1 μg/μL. The dilutions were picked up onto the tip of a stainless steel pin having a diameter of 0.5 mm cramped in a vertical position with freedom of vertical movement and spotted on the present substrate A and the comparative substrate by transferring them from the tip of the stainless steel pin to the surface of a substrate upon contact between them. The tip of the stainless steel pin was dipped in a dilution before each spot was formed. The dilutions were deposited in an amount of from 0.5 to 1.5 nL/spot.

The substrates were left still until the spots dried, and the fluorescence from the spotted surfaces at 575 nm and 670 nm was observed with a fluorometer under excitation lights of 532 nm and 635 nm. The fluorescence intensity from the present substrate A was higher than that from the comparative substrate and even over all the spots.

### Example 2

The procedure in Example 1 was followed except that instead of the substrate A, a present substrate B consisting of a soda lime glass plate having a thickness of 1 mm, a flatness of less than 50 μm and a refractive index of 1.5 and a TiNₓ coating having a thickness of about 40 nm, a surface roughness Ra of 30 nm, a refractive index of 2.5 and a surface resistance of 2×10¹⁰ Ω/□ formed by sputtering using a Ti target in a gas mixture of Ar and N₂ (at a gas flow rate of 30±10 sccm) at a pressure of 133 μPa, a power input of 0.26±0.1 kw and a traveling speed of 2.42±0.5 mm/s without heating the substrate was used, and no comparative substrate was used. The fluorescence was measured, and the fluorescence intensity was high and even over all the spots.

### Example 3

The procedure in Example 2 was followed except that a present substrate C prepared by forming a Cr film having a thickness of about 100 nm and a surface resistance of 5×10¹⁰ Ω/□ by sputtering using a Cr target instead of the Ti target under the same sputtering conditions as in Example 2 except that the gas mixture of Ar and N₂ was changed to Ar gas, and then forming on the Cr film, a SiO₂ film having a thickness of about 10 to 20 nm by sputtering using a Si target in O₂ gas (at a gas flow rate of 30±10 sccm) at a pressure of 133 μPa, a power input of 0.26±0.1 kw and a traveling speed of 2.09±0.5 mm/s without heating the substrate was used. The fluorescence was measured, and the fluorescence intensity was high and even over all the spots.

### Example 4

The procedure in Example 3 was followed except that a present substrate D prepared by forming an aminosilane organic film having a thickness of about 5 nm on the SiO₂ film of the present substrate C by dipping the present substrate C in an aminosilane solution in methanol and then drying, was used instead of the present substrate C. The fluorescence was measured, and the fluorescence intensity was high and even over all the spots. The fluorescence intensity was higher in Example 4 in which an organic film was formed than in Example 3.

The biochip substrate of the present invention allows measurement of the fluorescence from DNA analytes at high S/N ratios and makes it possible to obtain accurate data. Especially, fluorescence from low density analytes expressed at low levels can be analyzed precisely. The high S/N ratios allow formation of microspots and provision of highly integrated biochips. Therefore, it is possible to provide biochips useful for genetic research and gene analysis.

The entire disclosure of Japanese Patent Application No. 2003-410803 (filed on Dec. 9, 2003) including specification, claims, drawings and summary are incorporated herein by reference in its entirety.

1. A biochip substrate for producing a biochip by immobilizing a biological high-molecular-weight oligomer, which substantially reflects fluorescence on the surface on which the oligomer is to be immobilized.

2. The biochip substrate according to claim 1, which consists essentially of a support and a coating formed on the surface of the support on which the biological high-molecular-weight oligomer is to be immobilized, wherein the coating comprises a reflective film which has a higher refractive index than the support.

3. The biochip substrate according to claim 2, wherein the support is made of glass or plastic.

4. The biochip substrate according to claim 2, wherein the reflective film has a refractive index of at least 1.6.

5. The biochip substrate according to claim 2, wherein the refractive index of the support and the refractive index of the reflective film differ by at least 0.2.

6. The biochip substrate according to claim 2, wherein the reflective film has a thickness of from 5 to 200 nm.

7. The biochip substrate according to claim 2, wherein the reflective film contains at least one member selected from the group consisting of TiO₂, Ta₂O₅, ZrO₂, Al₂O₃, Si₃N₄, TiNₓ (0<X≦1), Cr, Ag and Ge.

9. The biochip substrate according to claim 2, which further comprises an organic film having affinity for the biological high-molecular-weight oligomer on the reflective film.

10. The biochip substrate according to claim 9, which has a film composed mainly of SiO₂ between the reflective film and the organic film.

11. The biochip substrate according to claim 1, which consists essentially of a metal support and an organic film formed on the surface of the metal support on which the biological high-molecular-weight oligomer is to be immobilized.

12. A process for producing a biochip substrate, which comprises forming, on a support, a reflective film having a higher refractive index than the support by spreading or sputtering.

13. The process for producing a biochip substrate according to claim 12, which further comprises forming an organic film on the reflective film by dipping or spreading.

14. The process for producing a biochip substrate according to claim 13, which further comprises forming a film composed mainly of SiO₂ between the reflective film and the organic film.
